(19) United States
(12) Patent Application Publication
(10) Pub. No.: US 2024/0207177 A1
(43) Pub. Date: Jun. 27, 2024

(54) Solid Phase Carrier for Vapor Delivery

(71) Applicant: Michael Baker, Guelph (CA)

(72) Inventor: Michael Baker, Guelph (CA)

(21) Appl. No.: 18/088,564

(22) Filed: Dec. 24, 2022

(51) Int. Cl. A61K 9/00; A24F 40/20; A61K 31/05; A61K 31/352; A61K 31/60

(52) U.S. Cl. CPC A61K 9/0075; A24F 40/20; A61K 31/05; A61K 31/352; A61K 31/60

(57) ABSTRACT

This invention outlines a novel substrate for delivery various biologically active substances by the mechanism of vaporizing the active component followed by inhalation for blood stream uptake by the lungs. The substrate composition and structure is particularly beneficial as it does not produce any toxic combustion products, unlike most vaping compositions currently available.

This application claims benefit of U.S. Provisional Patent Application No. 63/293,687, which was filed in the U.S. Patent and Trademark Office on Dec. 24, 2021, the entire contents of which are incorporated herein by reference for all purposes.

### BACKGROUND OF INVENTION

Vaping is the process of heating a substance beyond the temperature which causes a vapor to form and then inhaling that vapor. The process is often utilized to deliver biologically active substances to the lungs for rapid absorption and delivery to the blood stream. Conventionally, this process is accomplished by using carrier liquids. The carrier liquid serves the purpose of both diluting the biologically active substance, as well as decreasing viscosity or dissolving the biologically active substance to a level which can be handled most effectively.

Vaping devices are currently optimized to reach temperatures that are most effective for vaporizing nicotine and cannabinoid molecules. At these temperatures other molecules such as carrier liquids can go through combustion. Combustion is the process of breaking down carbon molecules in the presence of oxygen catalyzed by heat. Combustion products can be unpredictable and as a result species produced can vary greatly. In some cases toxic products can be formed from otherwise safe molecules. This problem is ever present in the case of most conventional carrier liquids and diluents.

One of the first carrier liquids used in vaping was a nearly even ratio of vegetable glycerin (VG) and propylene glycol (PG). This carrier liquid system is very effective at diluting most biologically active substances but also has demonstrated creation of toxic combustion products during vaping process. Such toxic products include formaldehyde. (Jensen, R., Strongin, R. & Peyton, D. Solvent Chemistry in the Electronic Cigarette Reaction Vessel. Sci Rep 7, 42549 (2017).) Medium chain triglyceride (MCT) oil has also been used as a carrier liquid but it too produces toxic combustion products.

There have since been new methods of delivering cannabinoids by vaping which include the use of various terpene mixtures. Terpenes are naturally found in cannabis plants, however, terpenes are normally found in cannabis plants around 5% maximum concentration. In order to reduce viscosity of cannabinoid oils, terpenes have been utilized as high as 20% in some vaping substances. The reason for the high usage is to effectively reduce viscosity of the cannabinoid oils. There is evidence that terpenes, especially in higher concentrations, can produce some harmful substances as a result of combustion. Both benzene and methacrolein have been detected in toxic quantities in vapour resulting from products that utilize terpenes for this purpose. (Jiries Meehan-Atrash, Wentai Luo, and Robert M. Strongin ACS Omega 2017 2 (9), 6112-6117.) This demonstrates that almost any organic component subjected to vaporizing level heat will produce some combustion products.

Vaping is intended to provide a safer alternative to smoking, since smoking of plant material produces many toxic combustion products. Currently marketed vaping technologies do not completely achieve this goal. There is an argument to made that vaping products are safer than smoking, but there is strong evidence of inherent toxicity in vaping products, despite improvements in comparison to smoking. This invention provides a safer alternative to both smoking and other conventional vaping products that utilize liquid carriers. Removal of all carbon based materials allows for a vaping product which delivers biologically active substances without toxic carbon combustion products. By instead introducing biologically active substances embedded on a salt that combusts within the vaporization temperature range to CO2 and H2O, a safer alternative to smoking can be completely achieved. This would allow for the dilution and effective dissolution provided by carrier liquid systems while improving on safety by eliminating carbon combustion products.

The invention is not limited to current vaping biologically active substances. This invention can be utilized for any biologically active substance which can benefit from the fast absorption and blood stream delivery afforded by introduction to the lungs. This invention could also be used to deliver biologically active substances to the blood stream in instances where the substance is not effectively absorbed by the gut, speed to blood stream is an asset and/or where the convenience of vaping as a delivery could be of benefit.

### SUMMARY OF THE INVENTION

As outlined in the background section, there is a growing demand for safer vaping products. Safer vaping products use non-toxic carrier compounds and produce minimal combustion products when heated to the temperature of vaporization. This new vaping technology is intended to offer a safer vaping alternative.

The embodiment of this invention is a free flowing powder which is comprised of biologically active substance and a salt substrate. The salt substrate is carefully chosen to have a degradation temperature which is slightly lower than the vaporization temperature of cannabis oil. During the vaping process, the substrate breaks down while biologically active substance vapors are formed. The salt substrate is carefully chosen to have gas phase combustion products of only water and carbon dioxide.

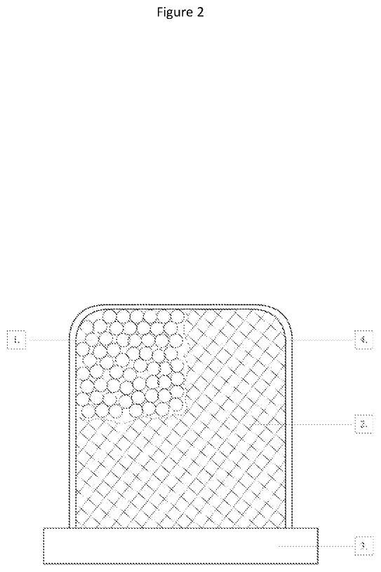

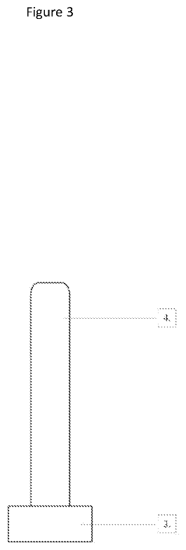

Another embodiment of this invention is the method of interfacing a solid vaping material with devices which are currently optimized for liquid vaping products. FIGS. 2-3 illustrate one method of introducing solid vaping material into the heated portion of a conventional vaping device. Additionally the solid substance could be pressed into a tablet.

Another embodiment of this invention is the method for producing the biologically active substance containing free flowing powder. This will ideally be accomplished by means of conventional processes in the industry for embedding substances onto a salt substrate.

### BRIEF DESCRIPTION OF DRAWINGS

The drawings are not necessarily drawn to scale. Like items in different drawings are numbered with the same numerals. This is a preferred adaptation enabling introduction of free flowing powder for the purposes of vaping into existing conventional vaping devices.

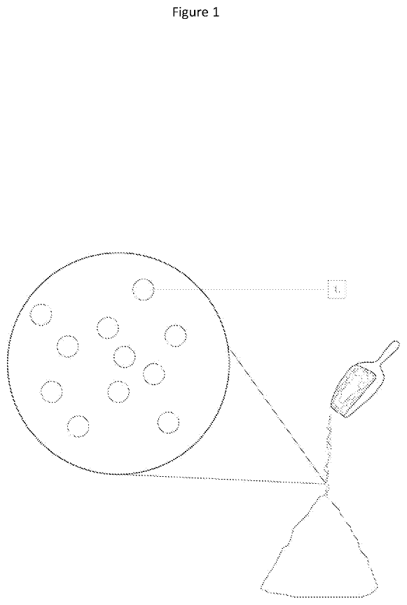

FIG. 1: Shows a free flowing powder demonstrating bulk properties
Item 1—powder particle

FIG. 2: Shows the ideal adaptation of introducing powder into conventional vaping device (front view)
Item 1—powder particle
Item 2—mesh screen to hold powder particle in place
Item 3—base of unit where screen and support are attached
Item 4—support for screen

FIG. 3: Shows the ideal adaptation of introducing powder into conventional vaping device (side view)
Item 3—base of unit where screen and support are attached
Item 4—support for screen

### DETAILED DESCRIPTION OF THE INVENTION

The general purpose of the invention is to provide a safe vaping product by eliminating liquid carrier substances and instead introducing biologically active substances to vaping devices by means of a free flowing powder or a tablet. This will be accomplished by embedding biologically active substances onto a salt substrate. The salt substrate will be carefully chosen to ensure that it too will form a gas phase product at desired vaporization temperatures, however, the salt will be carefully chosen to not form any combustion products other than CO2 and H2O. Introduction of the solid substance into a vaping device, will result in a biologically active substance rich vapor with minimal or even no toxic substances resulting from the vaping delivery mechanism.

The long term health effects of vaping have not yet been established as the technology is relatively new. The technology for vaping has only been on the market for about a decade and its significant current popularity has been realized for an even shorter time period. Acute health effects of vaping seem to have manifested during the recent vaping health crisis of summer 2019 and in comparison to smoking may in fact be worse. Deadly lung failure seems to be possible in cases which involve some combination of over consumption and potentially harmful carrier liquids. The health effects of vaping as a smoking alternative are not yet established fully and its current form may present a new set of health effects which are not observed with smoking.

The vaping process involves high temperature exposure to all molecules contained within the vaping device. When organic molecules are exposed to these temperature levels a process called combustion occurs. Combustion involves the breakdown of carbon molecules to water and carbon dioxide. When combustion produces only water and carbon dioxide this is distinguished as complete combustion. When complete combustion is not possible additional carbon molecules are left. These can be any combination of small carbon molecules, often including some very toxic carbons molecules such as formaldehyde. The carbon molecules formed and their resulting toxicity is a muti-variable process which cannot simply be summarized, but can accurately be described as potentially harmful to health in the best approximations.

This invention has applications which are not confined to current vaping applications. The invention is intended to provide novel delivery of any biologically active substance which can be vaporized at current vaping temperatures. This includes but is not limited to; cannabinoids, nicotine, caffeine, pharmaceutical active ingredients, natural extracts, terpenes, vitamins, narcotics or controlled substances. The invention includes synthetic or naturally derived substances. The invention is more broadly intended to provide vaporized biologically active substances to the lungs in order to benefit from the lungs high surface area and speed of which substances reach the blood stream. An additional benefit in some applications could be the ability to bypass the gut and liver. This invention can provide a safe and fast acting method of introducing biologically active substances to the blood stream.

Cannabinoid oils utilized in the invention can include any extracts from the cannabis plant. Extracts can be taken from any of Cannabis sativa, cannabis indica, Cannabis ruderalis or any hybrid combination of the plant species. These extracts can be prepared by any of the conventional means of purifying cannabinoids used in the industry. The cannabinoid oils are included in the composition between the concentrations of 0.1-50% by weight. The invention is intended to utilize either cannabinoid acids or cannabinoids that have been decarboxylated.

The invention utilizes either nicotine from natural sources or synthetic nicotine. Nicotine can be included in the invention in any of, but not limited to, the industry standard forms for vaping, including; high purity liquid, free base or nicotine salt. Nicotine has two enantiomeric forms, of which any proportional mix of the two forms can be used in the invention. Nicotine is included in the composition in concentrations of 0.1-50% by weight.

The invention is not limited to currently vaped biologically active substances such as cannabinoids and nicotine. Any biologically active molecule which can be vaporized and/or benefits from absorption through the lungs directly to the blood stream. The invention can also utilize any of, but not limited to, caffeine, pharmaceutical active ingredients, natural extracts, terpenes, vitamins, narcotics or controlled substances. These substances can be included in the invention in any combination with biologically active ingredients totaling 0.1-50% by weight.

Cannabinoid oils, and other pure substances, are very high in viscosity which makes working with them as pure substances prohibitive. The high viscosity will not allow for material flow during vaping process or packaging of the product. In order to remove this barrier, inherent in working with high purity substances, a diluent or carrier substance is utilized. Unlike in conventional vaping products, this invention instead utilizes a salt as a carrier substance. The salt serves to dilute the substances and provide physical properties that are more conducive to handling the product. This allows for the final embodiment of the invention to take on the form of a free flowing powder. Inclusion of a salt based carrier system allows for complete removal of all carbon based liquids, common in most vaping products, and as a result the toxic substances formed by carbon based material combustion are also eliminated.

Many substances intended for this invention are very potent when in their pure form. A safe and effective dose of pure substance is often in the milligram scale. This amount of substance is too difficult to handle and administer accurately. Thus another functional gain provided by the invention is the dilution of the biologically active substance to ensure the total dose is a easy to handle unit. Often an easy to handle does is on the scale of grams.

Ideally sodium bicarbonate would be utilized as the carrier substance. Sodium bicarbonate is ideal for many reasons. Sodium bicarbonate is non-toxic, non-reactive in most cases and an abundant cost effective material. Sodium bicarbonate is a substance that undergoes decomposition at temperatures above 176° F. (80° C.). Average vaping temperatures vary between 320° F. to 425° F. (160° C. to 218° C.), ensuring the sodium bicarbonate will break down and allow for maximum efficiency of vaporizing the target substance. At these temperatures sodium bicarbonate breaks down into three components—carbon dioxide (CO2), water (H2O) and disodium carbonate (Na2CO3). The disodium bicarbonate remains as a solid and is not part of the vapour generated. Therefore, by having sodium bicarbonate as the substrate for the powderized composition, when subjected to average vaping temperatures this composition will only contribute CO2 and H2O to the vapour consumed. There is a wide variety of salts which may provide a stable non-reactive and non-toxic substrate, but may provide less efficient vaporization mechanics due to less decomposition of the substrate at conventional vaping temperatures. The salt component can be between 50-99% of the powderized composition.

A binding agent is likely necessary to achieve maximum flow of the powder. Any industry standard binding agent could be used to accomplish this including but not limited to microcrystaline cellulose (MCC), carboxymethylcellulose (CMC), hydroxypropyl methyl cellulose (HPMC), hydroxypropyl cellulose (HPC), sodium carboxy methyl cellulose or polyvinyl pyrrolidone (PVP). FIG. 1 demonstrates a free flowing powder. A binding agent may be utilized within the range of 0.5 to 10% by weight. The zoomed circle in FIG. 1 displays that the powder is comprised of small particles identified as item 1 in drawings. MCC would ideally be used for this invention as it is the industry standard for binding agents and has a melting temperature above 500° F. (260° C.). It is also tasteless and has no smell. Therefore, it would not become part of the vaporized composition or experience, under standard vaping temperatures.

The powderized composition can be handled and introduced into vaping devices in many ways. For convenient adaptation to existing vaping device designs the powder could be pressed into a tablet, wafer, puck or any other shape or size which is optimal for vaping device designs. The invention has also improved upon liquid carrier systems by improving handling of the vaping substance. A flowing powder is much easier to handle, store and process than a viscous liquid. There stands to be measurable improvements in manufacturing time, manufacturing yields and manufacturing accuracy when using a powderized composition in favour of liquids.

Manufacture of a powderized composition can be accomplished by any industry standard methods of embedding thermally sensitive lipophillic substances onto a solid substrate. This includes but is not limited to spray drying, lyophilization, fluid bed drying, microencapsulation or other industry standard process. The ideal manufacturing method would yield a powderized free flowing substance with total solvent removal. The particle size can range from 0.0004 in to 0.12 in (10 μm to 3000 μm). The smaller the size of particles would provide for faster vapour generation by yielding increased surface area to volume of the particle. If introduced to vaping device with the adaptation described in FIG. 2 and FIG. 3, the particles would need to be larger than the mesh size of the air permeable screen, identified as 2 in drawing labels.

The powderized substance is to be introduced to vaping device with an adaptation of the device which allows for air flow around the powderized material and uniform exposure to heat source. The ideal interfacing of powder to vaping device would be as represented in FIG. 2 and FIG. 3. FIG. 2 shows the front of the adaptation. It is a small disposable unit which can range in size from 1 mm to 100 mm in the x or y dimensions on the plane of FIG. 2. Thus the surface area of item 2, in FIG. 2, can range from 0.015 in2 to 15.5 in2 (1 mm2 to 10000 mm2). FIG. 3 shows the side of the adaptation and exhibits the z axis of FIG. 2. The adaptation consists of a free flowing powderized composition, item 1, encased by an air permeable screen on either side of powder, item 2, supported a rigid material support, item 4, and both item 2 and item 4 are attached to a base from which the unit is handled, item 3. This adaptation to existing vaping devices will allow for convenient and easy use of the invention. This is an ideal adaptation but not the only possible adaptation. Interfacing of the powderized composition is not limited to this adaptation. Any adaptation which allows for airflow over a powderized composition and uniform exposure to heat can be utilized to interface powderized composition with existing vaping device technology.

1. A powderized composition comprising a biologically active substance and salt substrate which is vaporized to deliver biologically active substance to the surface of the lungs and ultimately absorbed into the blood stream.

2. The composition of claim 1 wherein, the composition has one or more biologically active substance applied to a salt substrate for a final weight percentage of 0.1-50% biologically active substance in the final composition.

3. The composition of claim 1 wherein, biologically active substances have been extracted, by industry standard mechanisms, or synthesized by industry standard mechanisms.

4. The composition of claim 1 wherein, biologically active substances can include but are not limited to; cannabinoids (tetrahydrocannabinol (THC) (and all its isomers), tetrahydrocannabinolic acid (THCa) (and all its isomers), cannabidiol (CBD), cannabidiolic acid (CBDa), cannabinol (CBN), cannabinolic acid (CBNa), cannabichromene (CBC), cannabichromic acid (CBCa), cannabigerol (CBG), cannabigerolic acid (CBGa)), nicotine (high purity liquid, free base, nicotine salt, with any combinations of (R) and (S) enantiomers), caffeine, pharmaceutical active ingredients, natural extracts, terpenes, vitamins, narcotics or controlled substances.

5. The composition of claim 1 wherein, the salt substrate accounts for a final weight percentage of 50-99% substrate in the final composition.

6. The composition of claim 1 wherein, the salt substrate can include but is not limited to the following cations; ammonium, calcium, copper, iron, magnesium, potassium, pyridium, sodium or other non-toxic and non-reactive cation.

7. The composition of claim 1 wherein, the salt substrate can include but is not limited to the following anions; acetate, bicarbonate, bromide, carbonate, chloride, citrate, cyanide, fluoride, hydroxide, iodide, nitritrate, nitrite, oxide, phosphate, phosphite, hydrogen phosphate, dihydrogen phosphate, sulphate, hydrogen sulphate, sulpide, hydrogen sulphide, sulphite, hydrogen sulphite or other non-toxic and non-reactive anion.

8. The composition of claim 1 wherein, a binding agent accounts for a final weight percentage of 0.5%-10% binding agent in the final composition.

9. The composition of claim 1 wherein, the binding agent can be but is not limited to; microcrystaline cellulose (MCC), carboxymethylcellulose (CMC), hydroxypropyl methyl cellulose (HPMC), hydroxypropyl cellulose (HPC), sodium carboxy methyl cellulose or polyvinyl pyrrolidone (PVP).

10. A powderized composition which is manufactured by, but not limited to, one of the following industry standard methods of applying material to a salt substrate; spray drying, lyophilization, fluid bed drying, microencapsulation. The method is to be optimized to produce 0.0004 in to 0.12 in (10 μm to 3000 μm) particles with any morphology which allows for a free-flowing powder.

11. The method of manufacture of claim 10 wherein, the free-flowing powder can be further processed, although not required, to free flowing powder, a pressed tablet of any shape or size, a convenient manufactured adaptation as outlined in drawings FIG. 2 and FIG. 3 or other industry standard method.

12. A method of consumption which introduces the powderized substance to a heating device which heats the powderized substance to a temperature between 176-600° F. (80-315° C.) by any industry standard method of generating vapor, including but not limited to; resistive heating of a metal coil, heating chamber, heated gas flow.

13. The method of consumption of claim 12 wherein, the powderized substance is introduced to a vaping device by, but not limited to, one of the following methods; free flowing powder, a pressed tablet of any shape, a convenient manufactured adaptation as outlined in drawings FIG. 2 and FIG. 3 or other industry standard method.

14. The method of consumption of claim 12 wherein, manufactured adaptions to introduce powder to vaping device include, but are not limited to, manufacture with the following materials; naturally occurring minerals, stainless steel, ceramic, non-reactive metal compositions, heat resistant polymers, silicone, resin, natural polymers.
